(19) United States
(12) Patent Application Publication
(10) Pub. No.: US 2011/0105510 A1
(43) Pub. Date: May 5, 2011

(54) PROPHYLACTIC/AMELIORATING OR THERAPEUTIC AGENT FOR NON-ALCOHOLIC STEATOHEPATITIS

(76) Inventor: Hiroshi Ishikawa, Shizuoka (JP)

(21) Appl. No.: 12/997,901

(22) PCT Filed: Jun. 17, 2009
(86) PCT No.: PCT/JP2009/061031

§ 371 Date: Dec. 14, 2010

(30) Foreign Application Priority Data
Jun. 17, 2008 (JP) 2008-158535

(51) Int. Cl. A61K 31/5025; A61K 31/20; A61K 31/437; A61P 1/16

(52) U.S. Cl. 514/248; 514/560; 514/303

(57) ABSTRACT

Disclosed are: a safe and highly effective prophylactic/ameliorating or therapeutic agent for NASH; and a method for using the agent. The prophylactic/ameliorating or therapeutic agent for NASH comprises a combination of at least one component selected from the group consisting of ω3PUFAs, a pharmaceutically acceptable salt thereof and an ester thereof and a PDE4 inhibitor as active ingredients.

### TECHNICAL FIELD

The present invention provides a prophylactic/ameliorative or therapeutic agent for non-alcoholic fatty liver disease, for non-alcoholic steatohepatitis in particular, and a method for using the agent.

### BACKGROUND ART

A group of liver diseases occurring in those having no alcohol drinking histories, including such hepatic disorders as simple fatty liver, steatohepatitis, hepatic fibrosis and hepatocirrhosis, are collectively defined as non-alcoholic fatty liver disease (hereafter referred to as “NAFLD”) except for viral liver diseases, autoimmune liver diseases, and metabolic liver diseases such as hemochromatosis and Wilson's disease. NAFLD can be divided on the basis of hepatobiopsy (pathological findings) into two stages, namely, simple fatty liver thought generally to be of good prognosis and non-alcoholic steatohepatitis (hereafter referred to as “NASH”) of bad prognosis, the latter being regarded as a severer form of NAFLD. Pathologic conditions determined by hepatobiopsy to be NASH, such as inflammation, pimelosis, fibrosis or cirrhosis, and hepatocellular carcinoma, are not different from those otherwise caused, and many of the hepatitides which are not considered as an alcoholic hepatic disorder, viral hepatitis or drug-induced hepatic disorder are expected to be a pathologic condition belonging to NASH (see Non-Patent Literature 1).

It is said that 20% of the population suffers from NAFLD, and 3% from NASH, in the United States. Also in Japan, such diseases are relatively often encountered upon a general medical practice, with the frequency of NAFLD in examinees being 8%, and it is estimated that the frequency of NASH in adult Japanese is at least 0.5 to 1%. Based on the fact that 13 million male and 10 million female adult Japanese have BMIs of 25 or greater indicating their obesity, domestic NAFLD patients are estimated to be 5 to 6 million in number and NASH patients approximately 300 to 500 thousand. In addition, lipid metabolism abnormality, hypertension, hyperglycemia and metabolic syndrome (hereafter referred to as “MetS”), all as defined in the criteria for diagnosis of MetS, have incidences as a complication of NAFLD of about 50%, about 30%, about 30% and about 40%, respectively (see Non-Patent Literature 1), so that it is expected that NASH will increase in number of cases and spread through younger generations along with the increase in lifestyle disease cases in the future. Moreover, a clinical problem is offered by a partial progress of hepatitis to hepatocirrhosis, or even to hepatocellular carcinoma by the activation of stellate cells.

In “NASH•NAFLD no Shinryo Gaido (Guidelines for Diagnosis and Treatment of NASH and NAFLD)” of The Japan Society of Hepatology (Non-Patent Literature 1) reporting the effectiveness of the NASH treatment methods which have been attempted aiming at the amelioration of a variety of pathologic conditions, it is stated at the same time that no treatment method is established yet at present. Specific examples of the treatment methods as described comprise methods using: insulin sensitizers, including biguanides (metformin), and thiazolidine derivatives (pioglitazone, rosiglitazone) as a PPAR-γ agonist; antioxidants such as vitamins, betaines (choline derivatives), and N-acetylcysteine; antihyperlipidemic agents, such as fibrate drugs (PPAR-α agonists), HMG-CoA reductase inhibitors (statins), and probucol; liver protection drugs such as ursodeoxycholic acid and polyene phosphatidylcholine (EPL); and angiotensin II receptor antagonists such as losartan.

There are reports on the administration of icosapentaenoic acid (hereafter referred to as “EPA”) or fish oil to NASH and NAFLD patients. For instance, it is reported that ω-3 polyunsaturated fatty acids (hereafter referred to as “PUFAs”), to be more specific, a mixture of EPA ethyl (hereafter referred to as “EPA-E”) and ethyl docosahexaenate (hereafter referred to as “DHA-E”) can ameliorate hepatitis in patients with NAFLD (see Non-Patent Literature 2). According to a latest report by Tanaka et al., amelioration of NASH is revealed by administering EPA-E of high purity at a dose of 2700 mg/day for 12 months, observing aspartate aminotransferase (hereafter referred to as “AST”) or alanine aminotransferase (hereafter referred to as “ALT”) enzyme, giving assessments by inflammatory cytokines or oxidative stress markers, and conducting hepatobiopsy after the period of administration and observation (see Non-Patent Literature 3).

A phosphodiesterase 4 (hereafter referred to as “PDE4”) inhibitor inhibits PDE4, which specifically degrades cyclic adenosine monophosphate (hereafter referred to as “cAMP”) in the cell, so as to increase the cAMP level and suppress the induction of inflammatory cytokine production in immunocytes. Various PDE4 inhibitors are developed as therapeutic agents against bronchial asthma, ulcerative colitis, allergic dermatitis, dementia, and so forth. It is known that the PDE4 family comprise four isozymes, PDE4A through PDE4D. PDE4B is involved in the tumor necrosis factor α (hereafter referred to as “TNFα”) production in leucocytes, monocytes and macrophages that is triggered by lipopolysaccharides (hereafter referred to as “LPS”), and the LPS-triggered TNFα production is reduced in leucocytes derived from a PDE4B knockout mouse. On the other hand, it has already been found that the LPS-triggered TNFα production in leucocytes derived from a PDE4D knockout mouse does not differ from that in leucocytes from a wild type mouse. In addition, PDE4D is expressed much at a site in the central nervous system which is related to vomiting, and behavioral inhibition as an index of vomiting is observed in a PDE4D knockout mouse, that is to say, there is apprehension that PDE4 inhibitors have a side effect of causing vomiting or nausea. Therefore, a PDE4 inhibitor highly specific to PDE4B is being sought as an antiinflammatory agent less causing vomiting or nausea as a side effect (see Non-Patent Literature 4).

The pyrrolopyridazine derivatives represented by formula (I), pyrazolopyridine derivatives represented by formula (II), pharmaceutically acceptable salts thereof, and their prodrugs as described later have in vitro PDE4 inhibitory activities and TNFα production-suppressing activities, so that it is expectedly described that they may be effective against a diversity of diseases allegedly mediated by PDE4 or TNFα (chronic inflammatory diseases (e.g., articular rheumatism, osteoarthritis, edema, emphysema, chronic bronchiolitis, allergic rhinitis), osteoporosis, rejection due to transplantation, asthma, chronic obstructive pulmonary disease (COPD), eosinophilia, fibrous diseases (e.g., cystic fibrosis, pulmonary fibrosis, hepatic fibrosis, renal fibrosis), (viral, alcoholic, drug-induced) acute, and fulminant hepatitis, fatty liver (alcoholic, and non-alcoholic steatohepatitis), chronic (viral, and non-viral) hepatitis, hepatocirrhosis, autoimmune hepatitis, pancreatitis, nephritis, endotoxic shock, certain autoimmune diseases [e.g., ankylosing spondylitis, autoimmune encephalomyelitis, autoimmune hematopathy (e.g., hemolytic anemia, hypoplastic anemia, erythroblastic aplasia, idiopathic thrombocytopenia), systemic lupus erythematosus (SLE), polychondritis, scleroderma, Wegener's granulomatosis, dermatomyositis, chronic active hepatitis (e.g., Wilson's disease), myasthenia gravis, idiopathic sprue, autoimmune inflammatory intestinal diseases (e.g., ulcerative colitis, Crohn's disease), endocrine ophthalmopathy, Graves' disease, sarcoidosis, multiple sclerosis, primary biliary hepatocirrhosis, juvenile diabetes (type 1 diabetes), Reiter's syndrome, non-infective uveitis, autoimmune keratitis (e.g., keratoconjunctivitis sicca, vernal conjunctivitis), interstitial lung fibrosis, psoriatic arthritis], dermatopathy associated with PDE4 enzyme (e.g., psoriasis and other benign or malignant proliferative dermatosis, atopic dermatitis, urticaria), neurodegenerative disorders (e.g., Parkinson's disease, Alzheimer's disease), acute, and chronic multiple sclerosis, cancerous cachexia, viral infection, AIDS cachexia, thrombosis, depression). There, however, are no descriptions on a proof of effectiveness gained in, or a specific application method used for, an actual patient with NASH or an actual animal model for NASH (see Patent Literatures 1 and 2).

As a system of combined application of drugs, it is proposed that a fibrate or a thiazolidine derivative and ω-3 PUFAs be applied in combination to the treatment of fatty liver associated with NASH or the like (see Patent Literature 3). Problems with the system are an excess PPAR-α activity in the case of using a fibrate, hepatotoxicity exerted in the case of using a thiazolidine derivative, and the aggravation of fatty liver by PPAR-γ upon basic testing. The application of ω-3 PUFAs and another drug in combination for the purpose of treating NAFLD or NASH is only known from Patent Literature 1.

### CITATION LIST

### Patent Literature

Patent Literature 1: WO 2004/063197
Patent Literature 2: WO 2006/004188
Patent Literature 3: WO 2007/081773

### Non-Patent Literature

Non-Patent Literature 1: The Japan Society of Hepatology ed., “NASH•NAFLD no Shinryo Gaido (Guidelines for Diagnosis and Treatment of NASH and NAFLD),” BUNKODO CO., LTD., Aug. 22, 2006.
Non-Patent Literature 2: Alimentary Pharmacology & Therapeutics, Vol. 23, No. 8, pp. 1143-1151, Apr. 15, 2006.
Non-Patent Literature 3: Journal of Clinical Gastroenterology, Vol. 42, No. 4, pp. 413-418, 2008.
Non-Patent Literature 4: Journal of Pharmacological Sciences (Folia Pharmacologica Japonica), Vol. 26, No. 2, pp. 121-127, 2005.

### SUMMARY OF INVENTION

### Technical Problems

An object of the present invention is to provide a prophylactic/ameliorative or therapeutic agent for NASH to be used for the prevention/amelioration or treatment of NAFLD, NASH in particular, and the suppression of progress to hepatocirrhosis/hepatocellular carcinoma as a severer condition, which agent is highly safe and effective as well as easy to use, and a method of using such an agent.

### Solution to Problems

The inventor of the present invention concentrated on researches in order to achieve the above object, and found at last that the application of ω-3 PUFAs and a PDE4 inhibitor in combination brings about such a safety as is not achieved with either drug as administered alone, and superb effects as well, so as to complete the present invention. In other words, the prophylactic/ameliorative or therapeutic agent for NAFLD or NASH as provided by the present invention comprise as active ingredients ω-3 PUFAs and a PDE4 inhibitor, preferably an inhibitor of high specificity to PDE4, more preferably an inhibitor of high specificity to PDE4B, especially at least one compound selected from the group consisting of pyrrolopyridazine derivatives represented by formula (I) as mentioned below, pyrazolopyridine derivatives represented by formula (II) as mentioned below, pharmaceutically acceptable salts thereof, and their prodrugs. The following are exemplary embodiments of the present invention.

(1) A prophylactic/ameliorative or therapeutic agent for NASH, in which at least one selected from the group consisting of ω-3 PUFAs as well as pharmaceutically acceptable salts and esters thereof, and a PDE4 inhibitor are applied in combination as active ingredients.

(2) The prophylactic/ameliorative or therapeutic agent according to (1) as above, wherein the ω-3 PUFAs as well as pharmaceutically acceptable salts and esters thereof consist of at least one compound selected from the group consisting of EPA, DHA, α-linolenic acid, as well as pharmaceutically acceptable salts and esters thereof.

(3) The prophylactic/ameliorative or therapeutic agent according to (1) as above, which contains EPA-E and/or DHA-E selected from among the ω-3 PUFAs as well as pharmaceutically acceptable salts and esters thereof.

(4) The prophylactic/ameliorative or therapeutic agent according to (1) as above, which contains EPA-E selected from among the ω-3 PUFAs as well as pharmaceutically acceptable salts and esters thereof.

(5) The prophylactic/ameliorative or therapeutic agent according to (1) as above, wherein the PDE4 inhibitor is at least one compound selected from the group consisting of pyrrolopyridazine derivatives represented by formula (I) as below, pyrazolopyridine derivatives represented by formula (II) as below, pharmaceutically acceptable salts thereof, and their prodrugs.

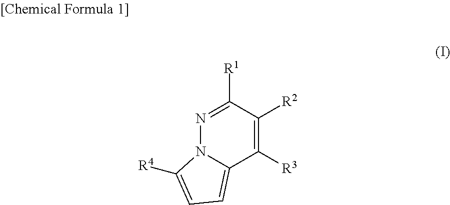

In formula (I),

R¹ is: (1) carboxy or protected carboxy; (2) —CONR⁵R⁶; (3) hydroxy or lower alkoxy; (4) mono- or di(lower)alkylamino optionally substituted with amino, cyclo(lower)alkylamino or lower alkoxy; (5) trihalo(lower)alkyl; (6) trihalo(lower)alkylsulfonyloxy or arylsulfonylamino; (7) substituted or unsubstituted lower alkyl; (8) substituted or unsubstituted aryl; or (9) a substituted or unsubstituted heterocyclic group,

R² is R⁷ or -(A¹)ₚ-X-A²-R⁷ [wherein p is 0 or 1, A¹ is (C₁-C₂) alkylene or —CH═CH—, A² is —(CH₂)ₙ—(n being any of integers 1 to 6) or —(CH═CH)ₘ— (m being any of integers 1 to 3), X is a single bond, —O—, —NR⁸—(R⁸ being hydrogen or lower alkyl), —C(═O)—, —C(═NR⁹)—(R⁹ being a substituted or unsubstituted N-containing heterocyclic group), or hydroxy(C₁-C₂)alkylene, and R⁷ is hydrogen; substituted or unsubstituted aryl; a substituted or unsubstituted heterocyclic group; carboxy, protected carboxy, or CONR¹⁰R₁₁; acyl or halocarbonyl; cyano; amino, protected amino, or mono- or di(lower)alkylamino; hydroxy, aryloxy, acyloxy, or lower alkyl optionally substituted with hydroxy or acyloxy; lower alkylthio, lower alkylsulfinyl, or lower alkylsulfonyl; or —O—R¹²], or

R¹ and R² form together a lower alkylene or lower alkenylene group, the group being optionally interrupted by amino or sulfonyl, or optionally condensed with a benzene ring, or optionally substituted with a group consisting of lower alkyl, hydroxy, oxo and lower alkoxy.

R³ is substituted or unsubstituted aryl, or a substituted or unsubstituted heterocyclic group, and R⁴ is hydrogen, halogen, cyano, carbamoyl, acyl, thiocyanate, lower alkylthio, lower alkenyl, hydroxy(lower)alkyl, trihalo(lower)alkyl, or lower alkyl.

R⁵, R⁶, R¹⁰, and R¹¹ are each independently hydrogen, lower alkylsulfonyl, a heterocyclic group, or lower alkyl optionally substituted with hydroxy, alkoxy, sulfo, carboxy, protected carboxy, or —R¹⁷; or R⁵ and R⁶, or R¹⁰ and R¹¹, along with a nitrogen atom to which they are bound, form a N-containing heterocyclic group, and R¹² and R¹⁷ are each independently a group derived from protected or unprotected sugar by removing a hydroxy group.

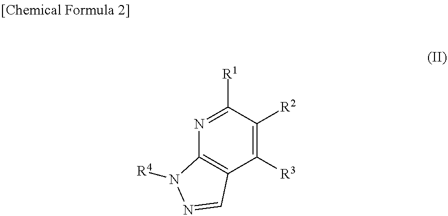

In formula (II),

R¹ is: (1) lower alkyl which is optionally substituted with halogen, cyclo(lower)alkyl, lower alkoxy, hydroxy, protected hydroxy, cyclo(lower)alkyloxy, aryloxy, hydroxyimino, carbamoyloxy optionally substituted with lower alkyl, or substituted or unsubstituted heterocyclyl (with the lower alkoxy being optionally substituted with cyclo(lower)alkyl, substituted or unsubstituted aryl, or substituted or unsubstituted heteroaryl); (2) lower alkenyl which is optionally substituted with cyano, or carbamoyl optionally substituted with aryl which may or may not contain halogen; (3) cyclo(lower)alkyl; (4) acyl; (5) cyano; (6) substituted or unsubstituted aryl; or (7) substituted or unsubstituted heteroaryl,

R² is R⁵ or -(A¹)ₚ-X-A²-R⁵ [wherein p is 0 or 1, A¹ is (C₁-C₂) alkylene or —CH═CH—, A² is a divalent heterocyclic group, or —(CH₂)ₙ—(n being any of integers 1 to 6) or —(CH═CH)ₘ— (m being any of integers 1 to 3), X is a single bond, —CH₂— or —O—, and R⁵ is hydroxy, protected hydroxy, cyano, acyl, carboxy, protected carboxy, hydroxyimino(lower)alkyl or —CONR⁶R⁷ [wherein R⁶ is hydrogen or lower alkyl, R⁷ is hydrogen or —(CH₂)_{q}—Y—R⁸ (wherein q is 0, 1, 2 or 3, Y is a bond, —O— or —CH(R⁹)—CH₂— (R⁹ being lower alkyl, carboxy, or protected carboxy), and R⁸ is lower alkyl; substituted or unsubstituted aryl; substituted or unsubstituted heteroaryl; substituted or unsubstituted heterocyclyl; or substituted or unsubstituted cyclo(lower)alkyl), or R⁶ and R⁷, along with a nitrogen atom to which they are bound, form a substituted or unsubstituted azaheterocyclyl group]],

R³ is: (1) substituted or unsubstituted aryl; (2) substituted or unsubstituted heteroaryl; (3) substituted or unsubstituted heterocyclyl; (4) cyclo(lower)alkyl; or (5) lower alkyl optionally substituted with (a) cyclo(lower)alkyl, (b) substituted or unsubstituted heterocyclyl, (c) substituted or unsubstituted aryl, or (d) substituted or unsubstituted heteroaryl, and

R⁴ is lower alkyl.

With respect to various definitions of the compounds to be included in the pyrrolopyridazinde derivatives represented by formula (I) and the pyrazolopyridine derivatives represented by formula (II), appropriate examples and exemplifications of the compounds, preferable forms thereof, such as substituents, pharmaceutically acceptable salts thereof, prodrugs, enantiomers or diastereoisomers, solvates, and radiolabeled derivatives, as well as preferable dosage routes, doses, dosage forms, excipients, and so forth, the contents of WO 2004/063197 and WO 2006/004188 can be referred to. The specifics as above are considered to be described in the present specification by citing the description of WO 2004/063197 for the pyrrolopyridazinde derivatives represented by formula (I), pharmaceutically acceptable salts thereof, and their prodrugs, and the description of WO 2006/004188 for the pyrazolopyridine derivatives represented by formula (II), pharmaceutically acceptable salts thereof, and their prodrugs, so that they are not illustrated herein.

(6) The prophylactic/ameliorative or therapeutic agent according to (1) as above, wherein the PDE4 inhibitor is at least one compound selected from the group consisting of 6-{4-[4-(aminocarbonyl)phenyl]-7-ethyl-2-methylpyrrolo[1,2-b]pyridazine-3-yl} hexanoic acid (hereafter referred to as “compound 1”), 4-(5-bromo-3-pyridyl)-1-ethyl-6-methyl-1H-pyrazolo[3,4-b]pyridine-5-carbonitrile (hereafter referred to as “compound 2”), (2E)-3-[1-ethyl-4-(5-methyl-3-pyridyl)-6-phenyl-1H-pyrazolo[3,4-b]pyridine-5-yl] acrylic acid (hereafter referred to as “compound 3”), (2E)-3-[4-(5-bromo-3-pyridyl)-1-ethyl-6-methyl-1H-pyrazolo[3,4-b]pyridine-5-yl] acrylic acid (hereafter referred to as “compound 4”), (2E)-3-[6-[(cyclohexylmethoxy)methyl]-1-ethyl-4-(5-methyl-3-pyridyl)-1H-pyrazolo[3,4-b]pyridine-5-yl] acrylic acid (hereafter referred to as “compound 5”), pharmaceutically acceptable salts thereof, and their prodrugs.

(7) The prophylactic/ameliorative or therapeutic agent according to (1) as above, wherein EPA-E and/or DHA-E is selected from among the ω-3 PUFAs as well as pharmaceutically acceptable salts and esters thereof, and the PDE4 inhibitor is at least one compound selected from the group consisting of compounds 1 through 5, pharmaceutically acceptable salts thereof, and their prodrugs.

(8) The prophylactic/ameliorative or therapeutic agent according to (1) as above, wherein EPA-E is selected from among the ω-3 PUFAs as well as pharmaceutically acceptable salts and esters thereof, and the PDE4 inhibitor is at least one compound selected from the group consisting of compounds 1 through 3, pharmaceutically acceptable salts thereof, and their prodrugs.

(9) The prophylactic/ameliorative or therapeutic agent according to any one of (1) through (8) as above, wherein the therapeutic effects of at least one selected from the group consisting of ω-3 PUFAs as well as pharmaceutically acceptable salts and esters thereof and at least one compound selected from the group consisting of pyrrolopyridazine derivatives represented by formula (I), pyrazolopyridine derivatives represented by formula (II), pharmaceutically acceptable salts thereof, and their prodrugs as applied in combination exceed the total therapeutic effects of ω-3 PUFAs and at least one compound selected from the group consisting of pyrrolopyridazine derivatives represented by formula (I), pyrazolopyridine derivatives represented by formula (II), pharmaceutically acceptable salts thereof, and their prodrugs as applied separately at the same doses as those upon the application in combination.

(10) The prophylactic/ameliorative or therapeutic agent according to any one of (1) through (9) as above, which is a composite formulation including at least one compound selected from the group consisting of ω-3 PUFAs as well as pharmaceutically acceptable salts and esters thereof and at least one compound selected from among PDE4 inhibitors.

(11) The prophylactic/ameliorative or therapeutic agent according to any one of (1) through (9) as above containing as an active ingredient at least one compound selected from the group consisting of ω-3 PUFAs as well as pharmaceutically acceptable salts and esters thereof, which is a prophylactic/ameliorative or therapeutic agent for NASH in the patient to whom a PDE4 inhibitor is administered.

(12) The prophylactic/ameliorative or therapeutic agent according to any one of (1) through (9) as above containing as an active ingredient at least one selected from among PDE4 inhibitors, which is a prophylactic/ameliorative or therapeutic agent for NASH in the patient to whom at least one selected from the group consisting of ω-3 PUFAs as well as pharmaceutically acceptable salts and esters thereof is administered.

(13) The prophylactic/ameliorative or therapeutic agent according to any one of (1) through (11) as above, in which at least one selected from the group consisting of ω-3 PUFAs as well as pharmaceutically acceptable salts and esters thereof and at least one selected from among PDE4 inhibitors are applied in combination by administering at least one compound selected from the group consisting of ω-3 PUFAs as well as pharmaceutically acceptable salts and esters thereof to the patient to whom a PDE4 inhibitor is administered.

(14) The prophylactic/ameliorative or therapeutic agent according to any one of (1) through (10) and (12) as above, in which at least one selected from the group consisting of ω-3 PUFAs as well as pharmaceutically acceptable salts and esters thereof and at least one selected from among PDE4 inhibitors are applied in combination by administering at least one selected from among PDE4 inhibitors to the patient to whom at least one compound selected from the group consisting of ω-3 PUFAs as well as pharmaceutically acceptable salts and esters thereof is administered.

(15) The prophylactic/ameliorative or therapeutic agent according to any one of (1) through (9) as above, which is a kit composed of separate formulations of at least one compound selected from the group consisting of ω-3 PUFAs as well as pharmaceutically acceptable salts and esters thereof and at least one selected from among PDE4 inhibitors.

(16) The prophylactic/ameliorative or therapeutic agent according to any one of (1) through (15) as above, in which at least one compound selected from the group consisting of liver protection drugs, hypoglycemic agents, antihyperlipidemic agents, antihypertensive agents, antioxidants, and antiinflammatory agents is further applied in combination as an active ingredient.

(17) A method for preventing/ameliorating or treating NASH, including the step of administering at least one selected from the group consisting of ω-3 PUFAs as well as pharmaceutically acceptable salts and esters thereof, and the step of administering a PDE4 inhibitor.

(18) The method according to (17) as above, wherein the two administering steps are implemented simultaneously.

(19) The method according to (17) as above, wherein the two administering steps are implemented at different timing.

(20) The method according to (17) as above, wherein administration is continued until values of at least one selected from the group consisting of the degree of hepatic fibrosis determined by an imaging test (e.g., echography, CT, MRI), hepatobiopsy, or from a fibrosis marker in the plasma (e.g., type IV collagen, hyaluronic acid, tissue inhibitor of metalloproteinases-1 (hereafter referred to as “TIMP-1”)), the serum AST or ALT level, the AST/ALT ratio, adiponectin, TNFα, interleukin (hereafter referred to as “IL”), high sensitivity C-reactive protein (hereafter referred to as “CRP”), the neutrophil count, and an oxidative stress marker in blood (ferritin, thioredoxin) fall within a normal range.

(21) The method according to any one of (17) through (20) as above, wherein the PDE4 inhibitor is at least one compound selected from the group consisting of pyrrolopyridazine derivatives represented by formula (I), pyrazolopyridine derivatives represented by formula (II), pharmaceutically acceptable salts thereof, and their prodrugs.

(22) A method for relieving side effects of at least one compound selected from the group consisting of pyrrolopyridazine derivatives represented by formula (I), pyrazolopyridine derivatives represented by formula (II), pharmaceutically acceptable salts thereof, and their prodrugs, including the step of administering at least one selected from the group consisting of ω-3 PUFAs as well as pharmaceutically acceptable salts and esters thereof, and the step of administering at least one compound selected from the group consisting of pyrrolopyridazine derivatives represented by formula (I), pyrazolopyridine derivatives represented by formula (II), pharmaceutically acceptable salts thereof, and their prodrugs.

(23) The method according to (22) as above, wherein the two administering steps are implemented simultaneously.

(24) The method according to (22) as above, wherein the two administering steps are implemented at different timing.

(25) The method according to (22) as above used for prevention/amelioration or treatment, wherein at least one selected from the group consisting of the reduction in dose of a PDE4 inhibitor, the withdrawal of a PDE4 inhibitor, and the increase in dose of ω-3 PUFAs is performed until possible vomiting or nausea ceases.

(26) The method according to any one of (22) through (25) as above, wherein the PDE4 inhibitor is at least one compound selected from the group consisting of pyrrolopyridazine derivatives represented by formula (I), pyrazolopyridine derivatives represented by formula (II), pharmaceutically acceptable salts thereof, and their prodrugs.

### Advantageous Effects of Invention

A safe and effective prophylactic/ameliorative or therapeutic agent for NASH and a method of using the agent are provided by a combined application of at least one selected from the group consisting of ω-3 PUFAs as well as pharmaceutically acceptable salts and esters thereof, and a PDE4 inhibitor, preferably an inhibitor of high specificity to PDE4, more preferably an inhibitor of high specificity to PDE4B, especially at least one compound selected from the group consisting of pyrrolopyridazine derivatives represented by formula (I), pyrazolopyridine derivatives represented by formula (II), pharmaceutically acceptable salts thereof, and their prodrugs.

Specifically, the agent as provided is expected to have synergistic effects of preventing/ameliorating or treating NASH as compared with the agent in which any of the drugs is applied alone. It is particularly predicted that the synergistic effects of preventing/ameliorating or treating NASH are found in the improvement in adipocytokine such as TNFα and IL, or high sensitivity CRP, the reduction in neutrophil count, and the improvement in fibrosis marker (type IV collagen, hyaluronic acid, TIMP-1 or the like) or oxidative stress marker in blood (ferritin, thioredoxin).

Vomiting or nausea, anorexia, or headache as an apprehended side effect of PDE4 inhibitors is intolerable to patients, and anorexia may cause malnutrition to adversely affect the amelioration of pathologic conditions. According to the present invention, the PDE4 inhibitor of high specificity to PDE4B can be used which is expected not to be so serious in side effect such as vomiting or nausea, and the dose of each drug, of the PDE4 inhibitor in particular, can be reduced, which allows relief from side effects such as vomiting or nausea, anorexia, or headache. In addition, treatment can be continued even for a patient in whom administration of a PDE4 inhibitor was heretofore not possible or could not help being discontinued due to side effects.

Moreover, by making the agent take the form of a composite formulation or a kit, the burden of medication on a patient is relieved, and the medication compliance of the patient is improved, leading to increased prophylactic/ameliorative or therapeutic effects.

### DESCRIPTION OF EMBODIMENTS

The present invention is detailed in the following.

The present invention provides a prophylactic/ameliorative or therapeutic agent for NASH, in which at least one selected from the group consisting of ω-3 PUFAs as well as pharmaceutically acceptable salts and esters thereof, and a PDE4 inhibitor, preferably an inhibitor of high specificity to PDE4, more preferably an inhibitor of high specificity to PDE4B, especially at least one compound selected from the group consisting of pyrrolopyridazine derivatives represented by formula (I), pyrazolopyridine derivatives represented by formula (II), pharmaceutically acceptable salts thereof, and their prodrugs, are applied in combination as active ingredients, and the method of using the agent. The prophylactic/ameliorative or therapeutic agent as provided by the present invention is the combined medicament for which at least one selected from the group consisting of ω-3 PUFAs as well as pharmaceutically acceptable salts and esters thereof, and a PDE4 inhibitor, especially at least one compound selected from the group consisting of pyrrolopyridazine derivatives represented by formula (I), pyrazolopyridine derivatives represented by formula (II), pharmaceutically acceptable salts thereof, and their prodrugs, are used in combination as active ingredients, with the method of using such a medicament being provided accordingly.

In the present invention, prevention should be construed not only as preventing the onset of a disease but delaying the onset and reducing the incidence rate.

In the present invention, amelioration should be construed as improving not only some parameter or other of a disease but the subjective symptoms or quality of life of a patient. In the present invention, treatment should be construed not only as administering a drug to the patient who has already developed a disease but administering a drug to a patient with a high risk of developing a disease as a prophylactic treatment.

Polyunsaturated fatty acids (PUFAs) are defined as those fatty acids each of which has a plurality of carbon-carbon double bonds in the molecule, and classified as ω-3 fatty acids, ω-6 fatty acids, and so forth in accordance with the positions of double bonds. Exemplary ω-3 PUFAs comprise α-linolenic acid, EPA, and docosahexaenoic acid (hereafter referred to as “DHA”). Unless otherwise specified, the term “PUFAs” as used herein implies not only polyunsaturated fatty acids but pharmaceutically acceptable salts as well as derivatives such as esters, amides, phospholipids and glycerides of polyunsaturated fatty acids.

The ω-3 PUFAs to be used in the present invention may be synthetic, semisynthetic or natural products, or may be in the form of natural oil containing them. The term “natural product” as used herein means a product obtained from a natural oil containing ω-3 PUFAs by a conventional extraction or crude purification, or a product obtained by highly purifying such a product. The term “semisynthetic product” implies a polyunsaturated fatty acid produced by a microorganism or the like, and also implies the polyunsaturated fatty acid as such or the polyunsaturated fatty acid as a natural product which has been subjected to a chemical treatment such as esterification or transesterification. In the present invention, a single ω-3 PUFA or a combination of two or more ω-3 PUFAs may be used.

The ω-3 PUFAs to be used in the present invention are specifically exemplified by EPA, DHA, α-linolenic acid, as well as pharmaceutically acceptable salts and esters thereof. Examples of pharmaceutically acceptable salts and esters comprise salts with inorganic bases such as sodium salt and potassium salt, salts with organic bases such as benzylamine salt and diethylamine salt, salts with basic amino acids such as arginine salt and lysine salt, as well as alkyl esters such as ethyl ester and esters of mono-, di- and triglycerides. Preferred is ethyl ester, especially EPA-E and/or DHA-E.

The ω-3 PUFAs are not particularly limited in purity, while it is generally preferable that the ω-3 PUFAs comprise not less than 25% by weight, more preferably not less than 50% by weight, and even more preferably not less than 70% by weight, especially not less than 85% by weight, of the fatty acids contained in the composition as the inventive agent. In a particularly desirable embodiment, the composition as the inventive agent contains essentially no other fatty acids than ω-3 PUFAs. In an exemplary case where EPA-E and DHA-E are to be used, the composition ratio EPA-E/DHA-E or the ratio of the (EPA-E+DHA-E) content to the total content of the fatty acids in the composition is not particularly limited, while the composition ratio EPA-E/DHA-E is preferably 0.8 or more, more preferably 1.0 or more, and even more preferably 1.2 or more. The combination of EPA-E and DHA-E is preferably of high purity, that is to say, as an example, the ratio of the (EPA-E+DHA-E) content to the total content of the fatty acids and derivatives thereof in the composition is preferably not less than 40% by weight, more preferably not less than 55% by weight, even more preferably not less than 84% by weight, especially not less than 96.5% by weight. In this connection, it is desirable that any long-chain saturated fatty acid content is low, and any ω-6 fatty acid, particularly arachidonic acid, is low in content even though it is a long-chain unsaturated fatty acid, whereupon a content lower than 2% by weight, in particular lower than 1% by weight, is preferred.

The EPA-E and/or DHA-E as used in the composition of the present invention is accompanied by less impurities unfavorable to cardiovascular events, such as saturated fatty acids and arachidonic acid, as compared with fish oils or concentrates thereof, and can exert effective actions without overnutrition or excess intake of vitamin A. In addition, the EPA-E and/or DHA-E, as being an ester, has a high oxidation stability as compared with the fish oils which are chiefly in the form of triglyceride, and allows a composition to be made adequately stable by adding a conventional antioxidant.

The EPA-E to be used may be in the form of high purity EPA-E (at least 96.5% by weight pure)-containing soft capsules available in Japan as a therapeutic agent against arteriosclerosis obliterans (ASO) and hyperlipidemia (trade name, EPADEL; manufactured by MOCHIDA PHARMACEUTICAL CO., LTD.). The mixture of EPA-E and DHA-E may be LOVAZA (manufactured by GlaxoSmithKline plc; soft capsules containing ca. 46.5% by weight EPA-E and ca. 37.5% by weight DHA-E) commercially available in the USA as a therapeutic agent against hypertriglyceridemia.

Purified fish oils may also be used as ω-3 PUFAs. Monoglycerides, diglycerides, triglycerides of ω-3 PUFAs, and combinations thereof are also comprised in preferable examples. A variety of commercially available products containing ω-3 PUFAs as well as salts and esters thereof, such as Incromega F2250, F2628, E2251, F2573, TG2162, TG2779, TG2928, TG3525 and E5015 (Croda International PLC, Yorkshire, England), and EPAX6000FA, EPAX5000TG, EPAX4510TG, EPAX2050TG, EPAX7010EE, K85TG, K85EE and K80EE (Pronova Biopharma, Lysaker, Norway), are also usable.

The PDE4 inhibitor to be used in the present invention may be any substance as long as it has PDE4 inhibitory activity, with the substance whose inhibitory activity is specific to PDE4 being preferred. A PDE4 inhibitor of higher specificity to PDE4B than to any other isozyme of PDE4, particularly having a high ratio of the intensity of PDE4B inhibitory activity to the intensity of PDE4D inhibitory activity, is preferable in terms of the relief from side effects such as vomiting or nausea. For instance, the PDE4 inhibitor has a ratio of the concentration for 50% PDE4B inhibitory activity to that for 50% PDE4D inhibitory activity of not more than 1, preferably not more than 0.1, more preferably not more than 0.05, and even more preferably not more than 0.01.

Examples of the PDE4 inhibitor comprise at least one compound selected from the group consisting of pyrrolopyridazine derivatives represented by formula (I), pyrazolopyridine derivatives represented by formula (II), pyrrolopyridazine derivatives represented by formula (I) which is shown in WO 2006/004191, pharmaceutically acceptable salts thereof, and their prodrugs, as well as doxofylline (Instituto Biologico Chemioterapico), roflumilast (ALTANA Pharma), tetomilast (Otsuka Pharmaceutical Co., Ltd.), cilomilast, denbufylline, AWD12-281, GSK1271836 and GSK256066 (GlaxoSmithKline), apremilast (Celgene Corp.), oglemilast (Glenmark Pharmaceuticals), piclamilast (sanofi-aventis), lilimilast and daxalipram (Bayer), ASP9831 (Astellas Pharma Inc.), OX-914 (Inflazyme Pharmaceuticals Ltd.), EHT-0202 (ExonHit Therapeutics), HT-0712 (Inflazyme Pharmaceuticals Ltd.), MEM-1414 (Memory Pharmaceuticals), arofylline (Almirall, S.A.), AN-2728 and AN-2898 (Anacor Pharmaceuticals, Inc.), HT-0712 and OX-914 (Inflazyme Pharmaceuticals Ltd.), SelCIDs, CC-1088 and CC11050 (Celgene Corp.), ONO-6126 and DE103 (ONO PHARMACEUTICAL CO., LTD.), GPD1116 (ASKA Pharmaceutical Co., Ltd.), ELB353 (elbion), GRC3886 and GRC4039 (Glenmark Pharmaceuticals), AVE-8112 (sanofi-aventis), 4AZA-PDE4 (4AZA Bioscience), CR-3465 (Rottapharm SpA), Rolipram (Schering AG), UCB-101333-3, CDP-840, L-791943, L-826141 and SCH351591 (UCB), RO 20-1724 and RS-25344-000 (Roche), KF19514 and KW4490 (Kyowa Hakko), PLX-369, PLX-377 and PLX-456 (Plexxikon, Inc.), CD-160130 (Curacyte AG), GP-0203 (Centre National de la Recherche Scientifique), INDUS-82010 (Indus Biotech Pvt. Ltd.), ND-1251 and ND1510 (Neuro3d SA), RBX-10017876 (Ranbaxy Laboratories), and CHF5480 (Chiesi Farmaceutici SpA). The inhibitor which particularly has a high ratio of the intensity of PDE4B inhibitory activity to the intensity of PDE4D inhibitory activity is exemplified by compounds 1 through 35 as pyrimidine derivatives described in Bioorg. Med. Chem. Lett. 19, pp. 3174-3176, 2009.

Preferred examples comprise at least one compound selected from the group consisting of pyrrolopyridazine derivatives represented by formula (I), pyrazolopyridine derivatives represented by formula (II), pyrrolopyridazine derivatives represented by formula (I) which is shown in WO 2006/004191, pharmaceutically acceptable salts thereof, and their prodrugs, as well as doxofylline, roflumilast, tetomilast, cilomilast, GSK256066, oglemilast, piclamilast, lilimilast, ASP9831, OX-914, EHT-0202, HT-0712, MEM-1414, AN-2728, AN-2898, CC11050, DE103, ELB353, and GRC4039, with ASP9831, and at least one compound selected from the group consisting of pyrrolopyridazine derivatives represented by formula (I), pyrazolopyridine derivatives represented by formula (II), pharmaceutically acceptable salts thereof, and their prodrugs being more preferred. Even more preferred examples comprise ASP9831, and at least one compound selected from the group consisting of compounds 1 through 5, pharmaceutically acceptable salts thereof, and their prodrugs, with at least one compound selected from the group consisting of compounds 1 through 3, pharmaceutically acceptable salts thereof, and their prodrugs being most preferred. Unless otherwise specified, the compounds as represented by formulae (I) and (II) that may be used in the present invention comprise such salts and prodrugs as mentioned above.

Compounds represented by formula (I) or (II) can be produced by a known method, the method as described in WO 2004/063197 or WO 2006/004188, for instance.

Of the inventive agent as described above, in which ω-3 PUFAs and a PDE4 inhibitor are applied in combination, a preferred embodiment is attained by a combined application of EPA-E and/or DHA-E and a compound represented by formula (I) or (II).

In the present invention, “application of active ingredients in combination” or “combined application of active ingredients” includes application of a combination of active ingredients, that is to say, administering ω-3 PUFAs and a PDE4 inhibitor as a formulation containing both, and administering ω-3 PUFAs and a PDE4 inhibitor as separate formulations simultaneously, or separately with a certain time lag, are included therein. In the mode of administration in which ω-3 PUFAs and a PDE4 inhibitor are administered “as separate formulations simultaneously, or separately with a certain time lag,” included are (1) administration of a composition containing a PDE4 inhibitor as an active ingredient to the patient to whom ω-3 PUFAs are to be administered, and (2) administration of a composition containing ω-3 PUFAs as active ingredients to the patient to whom a PDE4 inhibitor is to be administered. Moreover, although the drugs “applied in combination” are not necessarily limited to concurrently existing in the body of a patient, in the blood for instance, “application of active ingredients, or drugs, in combination” is defined in the present invention as the application method in which one drug is administered while the actions or effects of the other drug are exerted in the body of a patient. Such an application method makes it possible to prevent/ameliorate or treat a NAFLD- or NASH-associated disease effectively by using the prophylactic/ameliorative or therapeutic agent of the present invention. With respect to this application method, it is preferable that the drugs are concurrently present in the body of a patient, in the blood for instance, and that one drug is administered to a patient within 24 hours after the administration of the other.

In terms of the active ingredients of the prophylactic/ameliorative or therapeutic agent of the present invention, the mode of application in combination is not particularly limited as long as the active ingredients are combined with each other. The following are exemplary modes: (1) The active ingredients are formulated simultaneously, and the single formulation thus obtained is administered. (2) The active ingredients are formulated separately, and the two formulations thus obtained are combined together into a kit or kept separate, and administered simultaneously from one and the same dosage route. (3) The active ingredients are formulated separately, and the two formulations thus obtained are combined together into a kit or kept separate, and administered separately with a certain time lag from one and the same dosage route. (4) The active ingredients are formulated separately, and the two formulations thus obtained are combined together into a kit or kept separate, and administered simultaneously from different dosage routes (from different sites of one and the same patient). (5) The active ingredients are formulated separately, and the two formulations thus obtained are combined together into a kit or kept separate, and administered separately with a certain time lag from different dosage routes (from different sites of one and the same patient).

In the case of separate administration with a certain time lag, ω-3 PUFAs may be administered prior to a PDE4 inhibitor, or vice versa, for instance. In the case of simultaneous administration, the drugs may or may not be mixed together immediately before administration if the dosage route is one and the same. It is also possible to administer the drugs at different timing by design for various purposes. To be more specific: One drug may be administered and allowed to act when the effects of the other drug, which has previously been administered, begin to be exerted or are being fully exerted. Alternatively, one drug, a PDE4 inhibitor in particular, may be made into an extended release form to administer it once a day, whereupon the other drug, ω-3 PUFAs in particular, may be administered more than one time, two or three times for instance, daily, or also once a day. It is preferable that both drugs are administered once a day and, moreover, the drugs are administered simultaneously or as combined together into a single formulation because the burden of medication on a patient is relieved, and an improved medication compliance and increased prophylactic/ameliorative or therapeutic effects as well as reduced side effects are expected. The drugs may be administered simultaneously so as to stop administration of one drug when the effects of both drugs begin to be exerted or are being fully exerted. If the administration of a drug is to be stopped, the drug may gradually be reduced in dose. Administering one drug during the withdrawal of the other is also available.

In the prophylactic/ameliorative or therapeutic agent for NASH of the present invention, the method for application of ω-3 PUFAs and PDE4 inhibitors is not limited as long as the therapeutic effects of the inventive agent in which at least one ω-3 PUFA and at least one PDE4 inhibitor are applied in combination as active ingredients are not impaired. For instance, the inventive agent includes not only the prophylactic/ameliorative or therapeutic agent for NASH which is characterized by a sole application of ω-3 PUFAs and a PDE4 inhibitor, that is to say, which is composed of a combination of ω-3 PUFAs and a PDE4 inhibitor, but the prophylactic/ameliorative or therapeutic agent for NASH in which any further active ingredient is applied in combination.

It is desirable that the therapeutic effects of the ω-3 PUFAs and a PDE4 inhibitor as applied in combination exceed the total effects of the ω-3 PUFAs and a PDE4 inhibitor as applied separately at the same doses as those upon the application in combination. In this regard, the therapeutic effects are not particularly limited as long as they are effects of preventing/ameliorating or treating a NAFLD- or NASH-associated disease or suppressing progression thereof to hepatocirrhosis or hepatocellular carcinoma. Examples include the degree of hepatic fibrosis determined by an imaging test (e.g., echography, CT, MRI), hepatobiopsy, or from a fibrosis marker in the plasma (e.g., type IV collagen, hyaluronic acid, TIMP-1), the reduction in serum AST or ALT level, the reduction in AST/ALT ratio, the increase in adiponectin, the reduction in TNFα or IL, the reduction in high sensitivity CRP, the reduction in neutrophil count or reduction in oxidative stress marker in blood (ferritin, thioredoxin), and the improvement in HOMA-IR, with the improvement in adipocytokine such as TNFα and IL, or high sensitivity CRP, and the improvement in fibrosis marker (e.g., type IV collagen, hyaluronic acid, TIMP-1) or oxidative stress marker in blood (ferritin, thioredoxin) being preferred examples. Another biochemical or pathological parameter or pathologic condition parameter related to NAFLD or NASH may also be used to monitor prophylactic/ameliorative or therapeutic effects.

The doses and dosage timing of the ω-3 PUFAs and a PDE4 inhibitor used in the prophylactic/ameliorative or therapeutic agent of the present invention are made adequate to the expected actions of the drugs, and each modified as appropriate to the dosage form, dosage route, and frequency of administration per day of the relevant drug, the degree of a symptom, the body weight and age of a patient, and so forth.

In the case of oral administration, 0.1 to 10 g/day, preferably 0.3 to 6 g/day, more preferably 0.6 to 4 g/day, and even more preferably 0.9 to 2.7 g/day of EPA-E and/or DHA-E, for instance, is administered simultaneously or in two or three portions. Whether the entire amount is administered simultaneously or in portions may be determined as required. The dose may be reduced in response to the dose of a PDE4 inhibitor. Administration is preferably performed during meals or after meals, with an administration just after meals (within 30 minutes after a meal) being more preferred. The period of oral administration at the above dose will be at least one year, preferably two years or longer, more preferably 3.5 years or longer, and even more preferably 5 years or longer. It is desirable that administration be continued while a pathologic condition, biochemical index, or the like related to NASH remains, or the patient is under the situation where the risk of NASH onset and/or recurrence is great. Administration may also be performed every other day or two or three days a week, for instance, or with an optional drug withdrawal period for one day to about three months, preferably about one week to one month.

A PDE4 inhibitor for the prophylactic/ameliorative or therapeutic agent of the present invention is preferably used following the dosage regime for the relevant drug alone, while its dose may be modified as appropriate to the type, dosage form, dosage route, and frequency of administration per day of the drug, the degree of a symptom, the body weight, sexuality and age of a patient, and so forth. In the case of oral administration, 0.002 to 200 mg/day, preferably 0.02 to 20 mg/day, and more preferably 0.2 to 2 mg/day of compound 1, or 0.001 to 100 mg/day, preferably 0.01 to 10 mg/day, and more preferably 0.1 to 1 mg/day of compound 2 or 3, or 0.1 to 10000 mg/day, preferably 1 to 1000 mg/day, and more preferably 10 to 100 mg/day of cilomilast, or 0.002 to 200 mg/day, preferably 0.02 to 20 mg/day, and more preferably 0.2 to 2 mg/day of roflumilast, for instance, is administered simultaneously or in two portions. If necessary, the entire amount may be administered in several portions. In accordance with doctor's instructions, it is also possible to orally administrate the drug at a dose lower than the recommended daily dose on the first day of administration, then at a maintenance dose, with the dose being gradually increased up to the maximum daily dose. The dose may be reduced in response to the dose of ω-3 PUFAs. It is more preferable from the viewpoint of relief from side effects such as vomiting that the daily dose is reduced as much as possible, and an extended release formulation is utilized to achieve once-a-day administration. If a PDE4 inhibitor is orally administered with its dose as above, the dosage period will be at least one year, preferably two years or longer, more preferably 3.5 years or longer, and even more preferably 5 years or longer. It is desirable that administration be continued while a pathologic condition, biochemical index, or the like related to NASH remains, or the patient is under the situation where the risk of NASH onset and/or recurrence is great. Administration may also be performed every other day or two or three days a week, for instance, or with an optional drug withdrawal period for one day to about three months, preferably about one week to one month.

In the present invention based on the application of ω-3 PUFAs and a PDE4 inhibitor in combination, the dose of ω-3 PUFAs and/or a PDE4 inhibitor may be set lower than a conventional dose for general use. For instance, each drug may be used at a dose inadequate to gain therapeutic effects from the relevant drug alone. In that case, side effects of drugs, of a PDE4 inhibitor in particular, such as vomiting, are reduced with advantage.

If the dose of ω-3 PUFAs and/or a PDE4 inhibitor is inadequate to gain therapeutic effects from the relevant drug alone, it is also desirable that the therapeutic effects of the ω-3 PUFAs and the PDE4 inhibitor as applied in combination exceed the total effects of the ω-3 PUFAs and the PDE4 inhibitor as applied separately at the same doses as those upon the application in combination.

Again, if the dose of ω-3 PUFAs and/or a PDE4 inhibitor is inadequate to gain therapeutic effects from the relevant drug alone, it is also desirable that the side effects of the ω-3 PUFAs and the PDE4 inhibitor as applied in combination are reduced as compared with the total side effects of the ω-3 PUFAs and the PDE4 inhibitor as applied separately at the same doses as those upon the application in combination.

The dose of ω-3 PUFAs which is inadequate to gain therapeutic effects from them alone, as varying with the condition or habitus of each individual patient, is not limited, and is exemplified by the daily dose of EPA-E and/or DHA-E which is not less than 0.1 g but less than 2 g, and is preferably 0.2 to 1.8 g, more preferably 0.3 to 0.9 g, especially 0.3 to 0.6 g.

The dose of a PDE4 inhibitor which is inadequate to gain therapeutic effects from the drug alone, as varying with the condition or habitus of each individual patient, is not limited, and is exemplified by the daily dose of compound 1 which is less than 0.2 mg, preferably 0.002 to 0.15 mg, more preferably 0.005 to 0.1 mg, and even more preferably 0.01 to 0.05 mg, the daily dose of compound 2 or 3 which is less than 0.1 mg, preferably 0.001 to 0.08 mg, more preferably 0.002 to 0.05 mg, and even more preferably 0.005 to 0.02 mg, the daily dose of cilomilast which is less than 10 mg, preferably 0.1 to 8 mg, more preferably 0. 2 to 5 mg, and even more preferably 0.5 to 2 mg, and the daily dose of roflumilast which is less than 0.2 mg, preferably 0.002 to 0.15 mg, more preferably 0.005 to 0.1 mg, and even more preferably 0.01 to 0.05 mg.

The effects of the present invention are expected to be achieved with a PDE4 inhibitor at such a low dose as is below the dose at which the drug as applied alone exerts antiinflammatory action.

The dose ratio of ω-3 PUFAs to a PDE4 inhibitor is not particularly limited, while the preferred dose ratio of ω-3 PUFAs to a PDE4 inhibitor is exemplified by the dose ratio of ω-3 PUFAs to compound 1 which is 200-90000:1, preferably 300-30000:1, more preferably 500-10000:1, and most preferably 1000-6000:1, the dose ratio of ω-3 PUFAs to compound 2 or 3 which is 400-180000:1, preferably 600-60000:1, more preferably 1000-20000:1, and most preferably 2000-12000:1, the dose ratio of ω-3 PUFAs to cilomilast which is 4-1800:1, preferably 6-600:1, more preferably 10-200:1, and most preferably 20-120:1, and the dose ratio of ω-3 PUFAs to roflumilast which is 200-90000:1, preferably 300-30000:1, more preferably 500-10000:1, and most preferably 1000-6000:1. The dose of a PDE4 inhibitor may be reduced to one-half through one-tenth in order to relieve side effects of the PDE4 inhibitor. If a composite formulation is to be prepared, the drugs are desirably combined together at such a ratio as mentioned above.

With respect to a PDE4 inhibitor, as well as ω-3 PUFAs, the daily dose, the frequency of administration, or the dosage ratio can be modified appropriately by examining test values concerning the degree of hepatic fibrosis, the reduction in serum AST or ALT level, the reduction in AST/ALT ratio, the increase in adiponectin, the reduction in TNFα or IL, or reduction in neutrophil count, or reduction in oxidative stress marker in blood, the improvement in HOMA-IR, and so forth, or observing a patient on vomiting or nausea. As an example: The serum ALT level is measured when a PDE4 inhibitor is administered alone, then the measured value is used as an index to reduce the dose of the PDE4 inhibitor and start administration of ω-3 PUFAs, so as to achieve the therapeutic effects of the present invention. It is desirable that the side effects of the prophylactic/ameliorative or therapeutic agent of the present invention are at most comparable in frequency to the side effects, vomiting or nausea for instance, of the PDE4 inhibitor as administered alone at a dose required for the achievement of the same therapeutic effects as the present invention.

The active ingredient or ingredients of the prophylactic/ameliorative or therapeutic agent for NASH of the present invention may be administered as a compound (optionally including other constituents unremovable by purification) in itself, or combined with excipients suitably selected from among conventional carriers or media, vehicles, binders, lubricants, colorants, flavors, sterilized water or vegetable oils as required, as well as innoxious organic solvents or innoxious solubilizing agents (e.g., glycerin, propylene glycol), emulsifiers, suspending agents (e.g., Tween 80, gum arabic solution), isotonicities, pH-adjusting agents, stabilizers, soothing agents, corrigents, flavoring agents, preservatives, antioxidants, buffers, colorants, and the like, so as to prepare an appropriate medical formulation. The medical formulation may comprise as excipients, e.g., lactose, partially pregelatinized starch, hydroxypropylcellulose, macrogol, tocopherol, a hydrogenated oil, a sucrose ester of fatty acid, hydroxypropylmethylcellulose, titanium oxide, talc, dimethylpolysiloxane, silicon dioxide, carnauba wax or the like.

Since ω-3 PUFAs are of a highly unsaturated nature, it is particularly desirable to add an effective amount of an antioxidant, for instance, at least one selected from among butylated hydroxytoluene, butylated hydroxyanisole, propyl gallate, gallic acid, an pharmaceutically acceptable quinone, and α-tocopherol.

The dosage form of the formulation, as varying with the mode of combined application of active ingredients according to the present invention, is not particularly limited. The formulation may be administered to a patient orally, intravenously, intraarterially, by inhalation, rectally, intravaginally or externally, that is to say, as an oral formulation in the form of tablet, film-coated tablet, capsule, microcapsule, granule, fine granule, powder, oral liquid preparation, syrup, jelly, inhalant or the like, or as a parenteral formulation in the form of ointment, suppository, injection (emulsion, suspension, nonaqueous solution), solid injection to be emulsified or suspended before use, transfusion solution, external preparation such as endermic preparation, or the like. For those patients who are able to take oral formulations, easy-to-take oral formulations are desirable, so that oral administration of the formulation as encapsulated in a capsule such as soft capsule and microcapsule, in tablet form, or in film-coated tablet form is particularly preferred. It is also possible to administrate the formulation orally as an enteric preparation or an extended release preparation, or as a jelly in the case of dialysis patients or patients with dysphagia.

If two formulations prepared from ω-3 PUFAs and a PDE4 inhibitor, respectively, are combined with each other for use as the prophylactic/ameliorative or therapeutic agent of the present invention, the formulations are each prepared by a known method. The prophylactic/ameliorative or therapeutic agent of the invention may also be prepared as a composite formulation containing ω-3 PUFAs and a PDE4 inhibitor as active ingredients.

The composite formulation may further contain a third drug as an active ingredient. The third drug is not particularly limited, while preferable examples comprise those which do not weaken the effects of the present invention, such as a liver protection drug, a hypoglycemic agent, an antihyperlipidemic agent, an antihypertensive agent, an antioxidant, and an antiinflammatory agent.

The liver protection drug is exemplified by ursodeoxycholic acid and betaines. Examples of the hypoglycemic agent comprise insulin and insulin derivatives, slufonylurea drugs such as tolbutamide, gliclazide, glibenclamide and glimepiride, fast-acting insulin secretion stimulators such as nateglinide, repaglinide and mitiglinide, α-glucosidase inhibitors such as acarbose, voglibose and miglitol, thiazolidines such as pioglitazone, rosiglitazone and troglitazone, as well as biguanide-type hypoglycemic drugs such as metformin and buformin. Examples of the antihyperlipidemic agent comprise HMG-CoA reductase inhibitors such as pravastatin, simvastatin, atorvastatin, fluvastatin, pitavastatin, rosuvastatin and cerivastatin, fibrate drugs such as simfibrate, clofibrate, clinofibrate, bezafibrate and fenofibrate, lipase inhibitors such as orlistat, as well as ezetimibe. Examples of the antihypertensive agent comprise angiotensin-converting enzyme inhibitors such as captopril, alacepril, imidapril, enalapril, cilazapril, temocapril, delapril, lisinopril and benazepril, angiotensin II receptor antagonists such as losartan, valsartan, candesartan, telmisartan, olmesartan, irbesartan and eprosartan, renin inhibitors such as aliskiren, as well as calcium antagonists such as amlodipine, nifedipine, benidipine, nicardipine, nilvadipine, cilnidipine, azelnidipine, manidipine, nitrendipine, barnidipine, nisoldipine, efonidipine, felodipine, aranidipine, diltiazem, verapamil and bepridil. Examples of the antioxidant comprise vitamins such as vitamin C and vitamin E, N-acetylcysteine, and probucol. Examples of the antiinflammatory agent comprise cytokine production suppressors such as pentoxifylline, leukotriene receptor antagonists, leukotriene biosynthesis inhibitors, NSAIDs such as aspirin, COX-2 specific inhibitors, M2/M3 antagonists, steroids such as corticosteroid and prednisolone farnesylate, Hi (histamine) receptor antagonists, as well as aminosalicylates such as salazosulfapyridine and mesalazine. Exemplary immunosuppressants comprise azathioprine, 6-mercaptopurine, and tacrolimus. Exemplary antiviral agents against hepatitis C virus (HCV) comprise interferons, protease inhibitors, helicase inhibitors, and polymerase inhibitors.

The composite formulation is not particularly limited in dosage form, so that it is administered to a patient as an oral formulation in the form of tablet, film-coated tablet, capsule, microcapsule, granule, fine granule, powder, oral liquid preparation, syrup, jelly or the like, or as a parenteral formulation in the form of injection, transfusion solution, external preparation such as endermic preparation, or the like. In addition, the composite formulation comprise a formulation made adapted for extended release, a formulation releasing two drugs separately with a certain time lag, and so forth.

The composite formulation of the present invention may comprise a pharmaceutically acceptable vehicle in addition to active ingredients. The formulation may also contain a known antioxidant, coating agent, gelling agent, corrigent, flavoring agent, preservative, antioxidant, emulsifier, pH-adjusting agent, buffer, colorant or the like as appropriate.

The composite formulation of the present invention can be prepared according to a usual manner. Powder of ω-3 PUFAs is obtained by a known method in which, for instance, an oil-in-water emulsion containing (A) EPA-E, (B) dietary fiber, (C) a starch hydrolysate and/or a reducing starch decomposition product obtained by saccharification into oligosaccharide, and (D) a water-soluble antioxidant is dried in a high vacuum, then pulverized (JP 10-99046 A). By using the powder of EPA-E thus obtained and powder of a PDE4 inhibitor, a formulation in the form of granule, fine granule, powder, tablet, film-coated tablet, chewable tablet, extended release tablet, orally-disintegrating tablet (OD tablet) or the like can be prepared according to a usual manner. Chewable tablets may be obtained by the known method in which EPA-E is emulsified in a solution of water-soluble polymer such as hydroxypropylmethylcellulose, and the resultant emulsion is sprayed onto lactose or other excipient to form powdery glanules (JP 8-157362 A), with the granules being mixed with the powder of a PDE4 inhibitor for compressing. Extended release tablets may be obtained by (1) forming two layers containing EPA-E and a PDE4 inhibitor, respectively, so as to arrange one layer inside and the other outside, or (2) forming two matrix disks containing the two ingredients, respectively, so as to layer them, or (3) embedding particulate capsules including one ingredient into a matrix containing the other ingredient, or (4) mixing the two drugs together, then subjecting the mixture to some measures for extended release. It is desirable that the active ingredients are each regulated in releasing rate, and the two drugs may be released simultaneously or separately with a certain time lag. Orally-disintegrating tablets may be produced in accordance with such a known method as disclosed in JP 8-333243 A, and a film preparation for oral cavity may be produced in accordance with such a known method as disclosed in JP 2005-21124 A. Since many of PDE4 inhibitors are not simply soluble in ω-3 PUFAs, such measures as described in EXAMPLES should be taken if soft capsules or liquid preparation is chosen as the dosage form. Consequently, the composite formulation of the present invention comprises a formulation obtained by taking some measures or other to combining ω-3 PUFAs and a PDE4 inhibitor together into one formulation.

It is desirable that the active ingredients of the composite formulation of the present invention are so released and absorbed that their pharmacological actions may be exerted. Preferably, the composite formulation of the present invention has at least one effect out of an improved active-ingredient release, enhancement of the absorbability of active ingredients, enhancement of the dispersibility of active ingredients, an improved storage stability of the formulation in itself, and enhancement of the convenience to patients taking the formulation, or improvement of the compliance of such patients.

The prophylactic/ameliorative or therapeutic agent of the present invention is effective at preventing/ameliorating or treating NAFLD, NASH in particular, preventing recurrence thereof, or suppressing progression thereof to hepatocirrhosis or hepatocellular carcinoma in an animal, especially mammal. Exemplary mammals include humans, livestock animals such as cows, horses and pigs, as well as domestic animals such as dogs, cats, rabbits, rats and mice, with humans being preferred. The inventive agent is particularly expected to have synergistic effects of preventing/ameliorating or treating NASH on a patient with NASH presenting an increase in adipocytokine such as TNFα and IL, or high sensitivity CRP, in neutrophil count, or in fibrosis marker (e.g., type IV collagen, hyaluronic acid, TIMP-1) or oxidative stress marker in blood (ferritin, thioredoxin). The inventive agent makes it possible to relieve the side effects of PDE4 inhibitors such as vomiting, and continue treatment for a patient in whom administration of a PDE4 inhibitor was heretofore not possible or could not help being discontinued due to the side effects of the drug.

In addition, the prophylactic/ameliorative or therapeutic agent of the present invention as provided in the form of a composite formulation or a formulation kit relieves the burden of medication on a patient to improve the medication compliance of the patient, leading to further enhanced prophylactic/ameliorative or therapeutic effects.

### EXAMPLES

The present invention is illustrated in reference to the following examples, to which the present invention is in no way limited.

### Experimental Example 1

### Efficacy on Methionine-Choline-Deficient Diet Rat

Pharmacological actions of EPA-E and/or compound 3 on hepatic disorders and fibrosis are confirmed by using rats loaded with a methionine-choline-deficient diet (hereafter referred to as “MCD diet”), which are known for their development of NASH-like hepatic lesions.

Seven-week-old male Wistar rats are fed at 23° C. on a 12:12 light/dark cycle, whereupon they are permitted to take either an ordinary diet (F-1; Funabashi Farm Co., Ltd.) or a MCD diet (Dyets, Inc.) freely for 20 weeks. The rats are divided into five groups (each comprising 20 animals), namely, normal group (ordinary diet-loaded), control group (MCD diet-loaded), EPA-E group (MCD diet-loaded, plus EPA-E-administered), compound 3 group (MCD diet-loaded, plus compound 3-administered), and combined application group (MCD diet-loaded, plus EPA-E-administered, plus compound 3-administered). During the feeding, 1000 mg/kg of EPA-E is administered to the EPA-E group, 0.3 mg/kg of compound 3 to the compound 3 group, as well as 1000 mg/kg of EPA-E and 0.3 mg/kg of compound 3 are administered to the combined application group, with each administration being orally carried out once a day by suspending the relevant drug or drugs in a 5% aqueous solution of gum arabic. To the normal group and the control group, a 5% aqueous solution of gum arabic is orally administered once a day. After the feeding for 20 weeks, blood samples are collected for biochemical assays of the plasma, and pathological tests of the liver are conducted.

In the control group, the plasma AST, ALT, total bilirubin, albumin, total protein, cholinesterase, type IV collagen, hyaluronic acid and TIMP-1 levels, as well as the fibrotic area determined by Masson's trichrome stain and the hydroxyproline content of the liver are significantly increased as compared with the normal group, leading to the development of NASH-like hepatic lesions.

In the EPA-E group, increases of the plasma AST, ALT, total bilirubin, albumin, total protein, cholinesterase, type IV collagen, hyaluronic acid and TIMP-1 levels as well as the fibrotic area and the hydroxyproline content of the liver are suppressed as compared with the control group.

The compound 3 group is similar to the EPA-E group in effects achieved. In terms of the effects of suppressing increases of the above parameters, the therapeutic effects achieved in the combined application group exceed the total effects achieved in the EPA-E group and the compound 3 group. In consequence, the prophylactic/ameliorative or therapeutic agent of the present invention is useful for preventing/ameliorating or treating NASH, and so forth.

### Experimental Example 2

### Efficacy on Methionine-Choline-Deficient Diet Diabetic Mice

Pharmacological actions of EPA-E and/or compound 1 on hepatic disorders and fibrosis are confirmed by using diabetic mice loaded with a methionine-choline-deficient diet (hereafter referred to as “MCD diet”), which are known for their development of NASH-like hepatic lesions.

Seven-week-old male db/db mice (Charles River Japan, Inc.) are fed at 23° C. on a 12:12 light/dark cycle, whereupon they are permitted to take either an ordinary diet (F-1; Funabashi Farm Co., Ltd.) or a MCD diet (Dyets, Inc.) freely for two weeks. The mice are divided into five groups (each comprising 20 animals), namely, normal group (ordinary diet-loaded), control group (MCD diet-loaded), EPA-E group (MCD diet-loaded, plus EPA-E-administered), compound 1 group (MCD diet-loaded, plus compound 1-administered), and combined application group (MCD diet-loaded, plus EPA-E-administered, plus compound 1-administered). During the feeding, 1000 mg/kg of EPA-E is administered to the EPA-E group, 1 mg/kg of compound 1 to the compound 1 group, as well as 1000 mg/kg of EPA-E and 1 mg/kg of compound 1 are administered to the combined application group, with each administration being orally carried out once a day by suspending the relevant drug or drugs in a 5% aqueous solution of gum arabic. To the normal group and the control group, a 5% aqueous solution of gum arabic is orally administered once a day. After the feeding for two weeks, HOMA-IR measurement is performed, and blood samples are collected for biochemical assays of the plasma.

In the control group, HOMA-IR is aggravated, and the plasma AST, ALT, type IV collagen, hyaluronic acid and TIMP-1 levels are significantly increased as compared with the normal group.

In the EPA-E group, increases of the plasma AST and ALT levels are suppressed, and increases of the type IV collagen, hyaluronic acid and TIMP-1 levels are suppressed as compared with the control group.

The compound 1 group is similar to the EPA-E group in effects achieved. In terms of the effects of improving the above parameters, the therapeutic effects achieved in the combined application group exceed the total effects achieved in the EPA-E group and the PDE4 inhibitor group. In consequence, the prophylactic/ameliorative or therapeutic agent of the present invention is useful for preventing/ameliorating or treating NASH, and so forth.

### Experimental Example 3

### Efficacy on High Fat and High Sucrose Diet Taking Rats

Four-week-old male SD rats are fed at 23° C. on a 12:12 light/dark cycle, whereupon they are permitted to take either an ordinary diet (F-1; Funabashi Farm Co., Ltd.) or a high fat and high sucrose diet (TD88137 from Harlan Tekiad; hereafter referred to as “HF diet”) freely for four weeks. The rats are divided into five groups (each comprising 10 animals), namely, normal group (ordinary diet-loaded), control group (HF diet-loaded), EPA-E group (HF diet-loaded, plus EPA-E-administered), compound 2 group (HF diet-loaded, plus compound 2-administered), and combined application group (HF diet-loaded, plus EPA-E-administered, plus compound 2-administered). During the feeding, 1000 mg/kg of EPA-E is administered to the EPA-E group, 1 mg/kg of compound 2 to the compound 2 group, as well as 1000 mg/kg of EPA-E and 1 mg/kg of compound 2 are administered to the combined application group, with each administration being orally carried out once a day by suspending the relevant drug or drugs in a 5% aqueous solution of gum arabic. To the normal group and the control group, a 5% aqueous solution of gum arabic is orally administered once a day. After the feeding for four weeks, blood samples are collected to measure the neutrophil count and conduct biochemical assays of the plasma.

In the control group, the plasma AST and ALT levels are significantly increased, as well as the neutrophil count, TNFα, IL-6, and high sensitivity CRP are increased as compared with the normal group. Ferritin, thioredoxin, and type IV collagen are also increased.

In the EPA-E group and the compound 2 group, increases of the plasma AST and ALT levels are suppressed, and increases of the neutrophil count, TNFα, IL-6, high sensitivity CRP, ferritin, thioredoxin and type IV collagen are suppressed as compared with the control group.

In terms of the effects of improving the above parameters, the therapeutic effects achieved in the combined application group exceed the total effects achieved in the EPA-E group and the compound 2 group. In consequence, the prophylactic/ameliorative or therapeutic agent of the present invention is useful for preventing/ameliorating or treating NASH, and so forth.

### Experimental Example 4

Patients affirmatively diagnosed as having NASH are divided into three groups (each comprising 20 individuals), out of which the EPA-E group is caused to take EPADEL S (registered trademark) 900 (containing 900 mg of EPA-E) twice a day, the compound 3 group is caused to take a capsule containing 0.2 mg of compound 3 twice a day, and the combined application group is caused to take EPADEL S (registered trademark) 900 and a capsule containing 0.2 mg of compound 3, each twice a day. Compound 3 is initially administered once a day, that is to say, at a dose of 0.2 mg, and the dose is modified as appropriate to the patients' conditions within the range up to 0.4 mg, the daily dose if the capsule is taken twice a day, from the fifth week of administration onward. Following the method described in American Journal of Gastroenterology 2001, Vol. 96, pp. 2711-2717 with respect to the criteria for diagnosing patients, monitoring, histological testing, statistic analysis, and so forth, blood chemistry tests on ALT, AST, and the like are performed over a dosage period of one year, and hepatobiopsy is conducted at the end of dosage so as to make histological evaluations.

In the patients with NASH of any group, blood chemistry parameters such as the blood ALT or AST level are reduced from those before treatment. In addition, the liver tissue images under pathological examination are improved as compared with those before administration in the total evaluation of the grade of fat accumulation, the grade of inflammation, and the stage of fibrosis made in accordance with the method of Brunt. The individual indices are improved synergistically in the combined application group. Moreover, the dose of compound 3 is less increased, with the side effects of the compound such as vomiting being suppressed, in the combined application group as compared with the compound 3 group. In consequence, the prophylactic/ameliorative or therapeutic agent of the present invention is useful for preventing/ameliorating or treating NASH, and for relieving the side effects of compound 3 such as vomiting.

According to a usual manner, composite formulations are prepared from compounds 1 through 3 and EPA-E.

### Formulation Example 1

### Soft Capsules

TABLE 1
<Formulation Ex. 1 (soft capsules)>

| Ingredient | Unit amount | |
|---|---|---|
| EPA-E | 300 | mg |
| Compound 1 | 50 | μg |
| Gelatin | 170 | mg |
| D-Sorbitol | 25 | mg |
| Concentrated glycerin | 25 | mg |

| | |
|---|---|
| Sodium hydroxide | q.s. |
| Purified water | q.s. |

According to the composition as set forth in Table 1 above, concentrated glycerin, compound 1, and purified water are mixed with agitation, and the mixture is adjusted in pH to ca. 7 with sodium hydroxide. Gelatin and D-sorbitol are added to the resultant solution and dissolved therein by warming the solution with agitation to 60° C. Subsequently, the solution is degassed under a reduced pressure before its viscosity is modified with purified water to obtain a liquid material for soft capsule shells. The liquid material for soft capsule shells and EPA-E are used to prepare soft capsules each containing 300 mg of EPA-E and 50 μg of compound 1.

In a similar manner, soft capsules are prepared by using, instead of compound 1, 30 μg of compound 2, 20 μg of compound 3, 2.5 mg of cilomilast, or 50 μg of roflumilast per capsule.

### Formulation Example 2

### Soft Capsules

TABLE 2
<Formulation Ex. 2 (soft capsules)>

| Ingredient | | Unit amount | |
|---|---|---|---|
| A | EPA-E | 300 | mg |
| | Compound 1 | 100 | μg |
| B | Gelatin | 170 | mg |
| | D-Sorbitol | 25 | mg |
| | Concentrated glycerin | 25 | mg |

| | |
|---|---|
| Purified water | q.s. |

According to composition B as set forth in Table 2 above, water is mixed into concentrated glycerin, and then gelatin and D-sorbitol are added to the mixture and dissolved therein by warming the mixture with agitation to 60° C. The solution thus obtained is degassed under a reduced pressure before its viscosity is modified with purified water to obtain a liquid material for soft capsule shells. According to composition A as set forth in Table 2, pulverized compound 3 is mixed into EPA-E to obtain a uniform dispersion as a liquid content for soft capsules. The liquid material for soft capsule shells and the liquid content for soft capsules are used to prepare soft capsules each containing 300 mg of EPA-E and 100 μg of compound 1.

In a similar manner, soft capsules are prepared by using, instead of compound 1, 50 μg of compound 2, 50 μg of compound 3, 5 mg of cilomilast, or 100 μg of roflumilast per capsule.

### Formulation Example 3

### Liquid Preparation

TABLE 3
<Formulation Ex. 3 (liquid preparation)>

| Ingredient | | Unit amount | |
|---|---|---|---|
| A | EPA-E | 1800 | mg |
| | Orange oil | 81 | mg |
| B | Compound 1 | 500 | μg |
| | Polyoxyethylene (105) | 144 | mg |
| | polyoxypropylene (5) glycol | | |
| | Trehalose | 1350 | mg |
| | Ascorbic acid stearate | 1.8 | mg |
| | Sodium erythorbate | 117 | mg |

| | | |
|---|---|---|
| C | Sodium hydroxide | q.s. |
| | Purified water | q.s. |

| | | |
|---|---|---|
| Total | 9 | g |

The ingredients of composition B as set forth in Table 3 above are dissolved in purified water, with the resultant solution being adjusted in pH to ca. 7 with sodium hydroxide. The ingredients of composition A are added to the solution, which is then agitated at a high speed under a reduced pressure so as to make an emulsion. The emulsion is dispensed into stick packs made of an aluminum-laminated film so that each pack may contain 9 g of the emulsion. The packs are nitrogen-flushed and sealed to obtain a liquid preparation containing 1800 mg of EPA-E and 500 μg of compound 1 per pack.

In a similar manner, a liquid preparation is obtained by using, instead of compound 1, 200 μg of compound 2 or 100 μg of compound 3 per pack.

### Formulation Example 4

### Jelly Formulation

TABLE 4
<Formulation Ex. 4 (jelly formulation)>

| Ingredient | | Unit amount | |
|---|---|---|---|
| A | EPA-E | 1800 | mg |
| | Orange oil | 81 | mg |
| B | Compound 1 | 200 | μg |
| | Polyoxyethylene (105) | 144 | mg |
| | polyoxypropylene (5) glycol | | |
| | Trehalose | 1350 | mg |
| | Ascorbic acid stearate | 1.8 | mg |
| | Sodium erythorbate | 117 | mg |
| | Pullulan | 270 | mg |
| C | Carrageenan | 37.8 | mg |
| | Carob bean gum | 22.5 | mg |
| | Concentrated glycerin | 675 | mg |

| | | |
|---|---|---|
| D | Sodium hydroxide | q.s. |
| | Purified water | q.s. |

| | | |
|---|---|---|
| Total | 9 | g |

The ingredients of composition B as set forth in Table 4 above are dissolved in purified water, with the resultant solution being adjusted in pH to ca. 7 with sodium hydroxide. The ingredients of composition A are added to the solution, which is then agitated at a high speed under a reduced pressure so as to make an emulsion. The emulsion is warmed to 85° C., then, a uniform dispersion obtained by mixing the ingredients of composition C with agitation is added to the emulsion, with the whole being mixed to uniformity. The liquid preparation thus obtained is dispensed into stick packs made of an aluminum-laminated film so that each pack may contain 9 g of the preparation. The packs are nitrogen-flushed, sealed, and cooled to solidify their contents, so as to obtain a jelly formulation containing 1800 mg of EPA-E and 200 μg of compound 1 per pack.

In a similar manner, a jelly formulation is obtained by using, instead of compound 1, 100 μg of compound 2 or 50 μg of compound 3 per pack.

### INDUSTRIAL APPLICABILITY

The inventive prophylactic/ameliorative or therapeutic agent for NASH, in which at least one selected from the group consisting of ω-3 PUFAs as well as pharmaceutically acceptable salts and esters thereof, and a PDE4 inhibitor are applied in combination as active ingredients, is expected to have synergistic effects of preventing/ameliorating or treating NASH as compared with the agent in which any of the active ingredients is applied alone. The inventive agent is particularly expected to have synergistic effects of preventing/ameliorating or treating NASH on a patient with NASH presenting an increase in adipocytokine such as TNFα and IL, or high sensitivity CRP, in neutrophil count, or in fibrosis marker (e.g., type IV collagen, hyaluronic acid, TIMP-1) or oxidative stress marker in blood (ferritin, thioredoxin), such as a patient suffering from severer inflammation. In a patient with NASH suffering from milder inflammation also, pathologic conditions can be ameliorated significantly by applying the active ingredients as above in combination with a small amount of a HMG-CoA reductase inhibitor. According to the present invention, the PDE4 inhibitor of high specificity to PDE4B can be used which is expected not to be so serious in side effect such as vomiting or nausea, and the dose of each active ingredient can be reduced from that of the relevant ingredient as applied alone, which allows relief from the side effects of a PDE4 inhibitor in particular, such as vomiting. In addition, treatment can be continued even for a patient in whom administration of a PDE4 inhibitor was heretofore not possible or could not help being discontinued due to side effects.

Moreover, by making the agent take the form of a composite formulation or a kit, the burden of medication on a patient is relieved, and the medication compliance of the patient is improved, leading to increased prophylactic/ameliorative or therapeutic effects.

1. A prophylactic/ameliorative or therapeutic agent for non-alcoholic steatohepatitis, in which at least one selected from the group consisting of ω-3 polyunsaturated fatty acids as well as pharmaceutically acceptable salts and esters thereof, and a phosphodiesterase 4 inhibitor are applied in combination as active ingredients.

2. The prophylactic/ameliorative or therapeutic agent according to claim 1, wherein the phosphodiesterase 4 inhibitor comprises as an active ingredient applied in combination at least one compound selected from the group consisting of pyrrolopyridazine derivatives represented by formula (I): 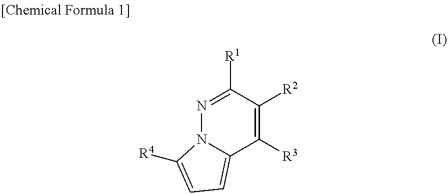
[in formula (I),
R¹ is:
(1) carboxy or protected carboxy;
(2) —CONR⁵R⁶;
(3) hydroxy or lower alkoxy;
(4) mono- or di(lower) alkylamino optionally substituted with amino, cyclo(lower) alkylamino or lower alkoxy;
(5) trihalo(lower) alkyl;
(6) trihalo(lower) alkylsulfonyloxy or arylsulfonylamino;
(7) substituted or unsubstituted lower alkyl;
(8) substituted or unsubstituted aryl; or
(9) a substituted or unsubstituted heterocyclic group,
R² is R⁷ or -(A¹)ₚ-X-A²-R⁷ [wherein p is 0 or 1, A¹ is (C₁-C₂) alkylene or —CH═CH—, A² is —(CH₂)ₙ— (n being any of integers 1 to 6) or —(CH═CH)ₘ— (m being any of integers 1 to 3), X is a single bond, —O—, —NR⁸— (R⁸ being hydrogen or lower alkyl), —C(═O)—C(═NR⁹)— (R⁹ being a substituted or unsubstituted N-containing heterocyclic group), or hydroxy(C₁-C₂) alkylene, and R⁷ is hydrogen; substituted or unsubstituted aryl; a substituted or unsubstituted heterocyclic group; carboxy, protected carboxy, or CONR¹⁰R¹¹; acyl or halocarbonyl; cyano; amino, protected amino, or mono- or di(lower) alkylamino; hydroxy, aryloxy, acyloxy, or lower alkyl optionally substituted with hydroxy or acyloxy; lower alkylthio, lower alkylsulfinyl, or lower alkylsulfonyl; or —O—R¹²], or
R¹ and R² form together a lower alkylene or lower alkenylene group, the group being optionally interrupted by amino or sulfonyl, or optionally condensed with a benzene ring, or optionally substituted with a group consisting of lower alkyl, hydroxy, oxo and lower alkoxy,
R³ is substituted or unsubstituted aryl, or a substituted or unsubstituted heterocyclic group,
R⁴ is hydrogen, halogen, cyano, carbamoyl, acyl, thiocyanate, lower alkylthio, lower alkenyl, hydroxy(lower)alkyl, trihalo(lower)alkyl, or lower alkyl,
R⁵, R⁶, R¹⁰, and R¹¹ are each independently hydrogen, lower alkylsulfonyl, a heterocyclic group, or lower alkyl optionally substituted with hydroxy, alkoxy, sulfo, carboxy, protected carboxy, or —R¹⁷; or R⁵ and R⁶, or R¹⁰ and R¹¹, along with a nitrogen atom to which they are bound, form a N-containing heterocyclic group, as well as R¹² and R¹⁷ are each independently a group derived from protected or unprotected sugar by removing a hydroxy group], pyrazolopyridine derivatives represented by formula (II): 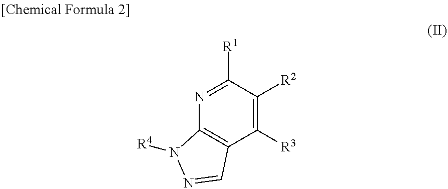
[in formula (II),
R¹ is:
(1) lower alkyl which is optionally substituted with halogen, cyclo(lower)alkyl, lower alkoxy, hydroxy, protected hydroxy, cyclo(lower)alkyloxy, aryloxy, hydroxyimino, carbamoyloxy optionally substituted with lower alkyl, or substituted or unsubstituted heterocyclyl (with the lower alkoxy being optionally substituted with cyclo(lower)alkyl, substituted or unsubstituted aryl, or substituted or unsubstituted heteroaryl);
(2) lower alkenyl which is optionally substituted with cyano, or carbamoyl optionally substituted with aryl which may or may not contain halogen;
(3) cyclo(lower)alkyl;
(4) acyl;
(5) cyano;
(6) substituted or unsubstituted aryl; or
(7) substituted or unsubstituted heteroaryl,
R² is R⁵ or -(A¹)ₚ-X-A²-R⁵ [wherein p is 0 or 1, A¹ is (C₁-C₂) alkylene or —CH═CH—, A² is a divalent heterocyclic group, or —(CH₂)ₙ— (n being any of integers 1 to 6) or —(CH═CH)ₘ— (m being any of integers 1 to 3), X is a single bond, —CH₉— or —O—, and R⁵ is hydroxy, protected hydroxy, cyano, acyl, carboxy, protected carboxy, hydroxyimino(lower)alkyl or —CONR⁶R⁷ [wherein R⁶ is hydrogen or lower alkyl, R⁷ is hydrogen or —(CH₂)_{q}—Y—R⁸ (wherein q is 0, 1, 2 or 3, Y is a bond, —O— or —CH(R⁹)—CH₂— (R⁹ being lower alkyl, carboxy, or protected carboxy), and R⁸ is lower alkyl; substituted or unsubstituted aryl; substituted or unsubstituted heteroaryl; substituted or unsubstituted heterocyclyl; or substituted or unsubstituted cyclo(lower)alkyl), or R⁶ and R⁷, along with a nitrogen atom to which they are bound, form a substituted or unsubstituted azaheterocyclyl group]], R³ is:
(1) substituted or unsubstituted aryl;
(2) substituted or unsubstituted heteroaryl;
(3) substituted or unsubstituted heterocyclyl;
(4) cyclo(lower)alkyl; or
(5) lower alkyl optionally substituted with (a) cyclo(lower)alkyl, (b) substituted or unsubstituted heterocyclyl, (c) substituted or unsubstituted aryl, or (d) substituted or unsubstituted heteroaryl, as well as R⁴ is lower alkyl], pharmaceutically acceptable salts thereof, and their prodrugs.

3. The prophylactic/ameliorative or therapeutic agent according to claim 1 or 2, wherein the ω-3 polyunsaturated fatty acids as well as pharmaceutically acceptable salts and esters thereof comprise at least one compound selected from the group consisting of icosapentaenoic acid, docosahexaenoic acid, α-linolenic acid, as well as pharmaceutically acceptable salts and esters thereof.

4. The prophylactic/ameliorative or therapeutic agent according to any one of claims 1 through 3, wherein at least one compound selected from the group consisting of pyrrolopyridazine derivatives represented by formula (I), pyrazolopyridine derivatives represented by formula (II), pharmaceutically acceptable salts thereof, and their prodrugs is at least one compound selected from the group consisting of 6-{4-[4-(aminocarbonyl)phenyl]-7-ethyl-2-methylpyrrolo[1,2-b]pyridazine-3-yl}hexanoic acid, 4-(5-bromo-3-pyridyl)-1-ethyl-6-methyl-1H-pyrazolo[3,4-b]pyridine-5-carbonitrile, (2E)-3-[1-ethyl-4-(5-methyl-3-pyridyl)-6-phenyl-1H-pyrazolo[3,4-b]pyridine-5-yl] acrylic acid, (2E)-3-[4-(5-bromo-3-pyridyl)-1-ethyl-6-methyl-1H-pyrazolo[3,4-b]pyridine-5-yl] acrylic acid, (2E)-3-[6-[(cyclohexylmethoxy)methyl]-1-ethyl-4-(5-methyl-3-pyridyl)-1H-pyrazolo[3,4-b]pyridine-5-yl] acrylic acid, pharmaceutically acceptable salts thereof, and their prodrugs.

5. The prophylactic/ameliorative or therapeutic agent according to any one of claims 1 through 4, which is a composite formulation comprising at least one compound selected from the group consisting of ω-3 polyunsaturated fatty acids as well as pharmaceutically acceptable salts and esters thereof, and at least one compound selected from the group consisting of pyrrolopyridazine derivatives represented by formula (I), pyrazolopyridine derivatives represented by formula (II), pharmaceutically acceptable salts thereof, and their prodrugs.
